(19) United States
(12) Patent Application Publication
(10) Pub. No.: US 2005/0124029 A1
(43) Pub. Date: Jun. 9, 2005

(54) Process for the preparation of a betha- lactam antibiotic with mutated penicillin acylase

(76) Inventors: Wynand Bernlef Liudger Alkema, Stockholm (SE); Harold Monro Moody, Gulpen (NL); Jan Metske Van Der Laan, AP Breda (NL); Theodorus Johannes Godfried Maria Van Dooren, BZ Roermond (NL); Wilhelmus Hubertus Joseph Boesten, BJ Sittard (NL)

Correspondence Address: MORRISON & FOERSTER LLP, 3811 VALLEY CENTRE DRIVE, SUITE 500, SAN DIEGO, CA, 92130-2332, US

(21) Appl. No.: 10/499,662

(22) PCT Filed: Dec. 18, 2002
(86) PCT No.: PCT/EP02/14610

(30) Foreign Application Priority Data
Dec. 27, 2001 (NL) 1019667

(51) Int. Cl. C12P 35/04; C12N 9/10; C07D 501/14; C12N 1/21

(52) U.S. Cl. 435/50; 435/193; 435/252.33; 540/222

(57) ABSTRACT

The invention relates to mutated penicillin acylases having a high synthesis/hydrolysis ratio in comparison with wild-type penicillin acylases and at least 1% of the activity. The invention also relates to a process for the enzymatic preparation of a β-lactam antibiotic from a β-lactam nucleus and an activated side chain with the aid of mutated penicillin acylases.

The invention relates to a mutated penicillin acylase and a process for the preparation of a β-lactam antibiotic wherein a β-lactam nucleus is acylated with the aid of an activated side chain in the presence of a mutated penicillin acylase.

Penicillin acylases are a group of hydrolases originating from microorganisms, for example bacteria, that are capable of reversibly hydrolyzing the 6-acyl group of penicillins or the 7-acyl group of cephalosporins to form the corresponding free amines without the ring structure of the penicillins or cephalosporins being destroyed. Reaction diagram I illustrates a hydrolysis reaction. A few examples of side chains R in the penicillin compound are phenylacetyl and phenoxyacetyl.

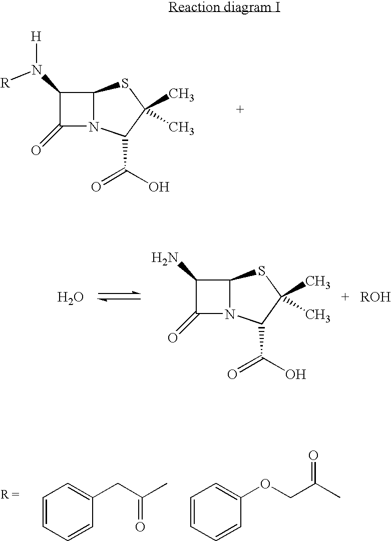

Penicillin acylases are variously known as for example penicillin amidase or benzyl penicillin hydrolase (enzyme classification E.C. 3.5.1.11).

The term β-lactam antibiotic comprises all antibiotics that contain a condensed ring system as shown by formula II or III, where X═S or O. The best-known β-lactam antibiotics are the penicillins and cephalosporins.

In the context of the present application penicillins are defined as compounds according to formula (II) and cephalosporins as compounds according to formule (III), 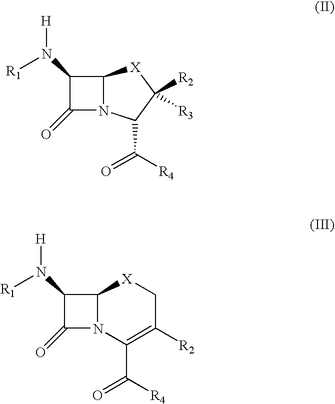
where
X═S, O, C, S(O), or SO₂,
R₁=side chain, such as for example phenylacetyl, phenoxyacetyl, hydroxyphenylglycyl, phenylglycyl, dihydrophenylglycyl and derivatives thereof and acetyl, adipyl, glutaryl and derivatives thereof,
R₂, R₃═Cl, aliphatic or aromatic groups, optionally with one or more O, S or N atoms,
R₄═OH, aliphatic or aromatic alcohols and derivatives thereof, optionally with one or more O, S or N atoms.

Side chains in the context of the present invention may include any suitable compounds that can be attached to the 6-penam or 7-cephem position of a β-lactam nucleus, resulting in an antibiotically active compound.

The aliphatic group in R₂or R₃ preferably contains 1-4 C atoms, and is preferably a methyl group.

Penicillins or cephalosporins are prepared for example by acylating the 6-amino-group of 6-aminopenicillanic acid (6-APA) or a derivative thereof as shown in formula (IV), or the 7-amino group of 7-aminodesacetoxycephalosporanic acid (7-ADCA) or a derivative thereof as shown in formula (V), with the aid of an activated side chain and a penicillin acylase enzyme. Also 7-aminocephalosporanic acid (7-ACA)-nuclei can be acylated with the aid of penicillin acylases. Derivatives of the compounds according to formula (IV) or (V) are understood to be compounds according to formula (IV) or (V), respectively, wherein X, R₂, R₃, and R₄ have the meaning shown in formula (II) and (III), respectively. 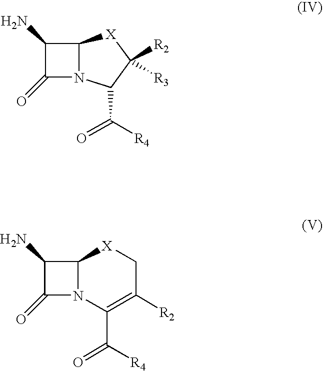
where
X═S
R₂═CH₃
R₃═CH₃
R₄═OH

Penicillin acylases can be classified both on the basis of their molecular structure and on the basis of their substrate specificity. There are type I, II and III acylases. Type II acylases consist of a heterodimer of a (small) α-subunit and a (large) β-subunit. Type IIa, the so-called penicillin G acylases, are active on substrates with a hydrophobic side chain such as for example phenylacetyl, the side chain of Penicillin G. Short alkyl chains, for example, are recognised also by Type IIa acylases. However, Type IIa acylases are not active with substrates with charged side chains. For substrates with charged side chains Type IIb acylases are suitable. All Type II acylases belong to one family. It is known that Type II acylases show high homology in amino acid sequence.

In the framework of this application penicillin acylase is understood to be a Type II acylase. In a preferred embodiment the invention relates to Type IIa penicillin acylases.

A mutated penicillin acylase or a penicillin acylase mutant is understood to be a penicillin acylase in which in at least one position an amino acid from the amino acid sequence of a wild type penicillin acylase has been replaced by another amino acid.

Mutants are described by the number of the position of the amino acid which is replaced in the amino acid sequence of the wild type. Before the number it is indicated which amino add occurs at that place in the wild-type, and after the number it is indicated which amino acid has taken its place in the mutant.

SEQ ID No:1 shows the amino acid sequence of an E. coli penicillin acylase, including secretion signal. SEQ ID No:2 shows the amino acid sequence of the α-subunit of an E. coli penicillin acylase. SEQ ID No:3 shows the amino acid sequence of the β-subunit of an E. coli penicillin acylase. In SEQ ID No:4 the amino acid sequence is given of the mutated α-subunit at position 145 of an E. coli penicillin acylase (α-R145L).

Numerous publications are known in which reference is made to mutated penicillin acylases. In WO98/20120 reference is made for example to the following publications: Prieto et al, Appl. Microbiol.Biotechnol.33 (1990) 553-559, in which it is described that replacement of L-methionine in position 168 in penicillin acylase of K. citrophila by L-alanine (M168A), L-valine (M168V), L-asparagine (M168N) or L-tyrosine (M168Y) had an influence on the kinetic parameters for deacylation of penicillin G and penicillin V, while the substitution of Asn375 or Tyr481 by lysine (N375K) and histidine (Y481H), respectively, had no effect. In J. Martin and I. Prieto, Biochimica et Biophysica Acta 1037 (1990) 133-139, a mutant is described in which Metl68 is replaced by alanine (M168A). This resulted in improved thermal stability. Replacement of Ser177 in E. coli penicillin acylase by glycine (S177G), threonine (S177T), leucine (S177L), or arginine (S177R) produced inactive enzymes (Wang Min et al., Shiyan Shengwu Xuebao 24 (1991) 1, 51, while from Kyeong Sook et al., Journal of Bacteriology 174 (1992) 6270 and Slade et al., Eur. J. Biochem.197, (1991)75 it is known that Ser290 is an essential amino acid in penicillin acylase of E. coli. Replacement of Gly359 by L-aspartic acid (G359D) resulted in a mutated enzyme which was not able to hydrolyze penicillin G but which was able to hydrolyze phthalyl-leucine and phthalyl-glycyl-L-proline. Replacement of Trp431 in Arg (W431R; Gabriel Del Rio et al., Biotechnology and Bioengineering 48 (1995) 141-148) resulted in a mutated penicillin G acylase of E. coli with increased stability in a basic environment.

A recent publication is W. B. L. Alkema et al., Protein Engineering, 13, (2000) 857-863, In which the binding site of E. coli penicillin acylase is characterised, and in which it is further described that a mutant with L-tyrosine on α146 (F146Y) was not active for the synthesis of penicillin G from phenylacetamide and 6-aminopenicillanic acid, while mutants with leucine (F146L) or alanine (F146A) at that position were more effective for the synthesis of penicillin G than the wild type.

Patents in which many mutants of penicillin acylases are described are European patent application EP-A-0453048 and international patent application WO 96/05318.

For an economically attractive process for the enzymatic preparation of a β-lactam antibiotic it is desirable that the Synthesis/Hydrolysis ratio (S/H ratio) is high. Preferably the S/H ratio Is high and at the same time the enzymatic activity is also sufficiently high.

The Synthesis/Hydrolysis ratio (S/H ratio) is understood to be the molar ratio of synthesis product to hydrolysis product at defined conditions during the enzymatic acylation reaction. Synthesis product is understood to be the β-lactam antibiotic formed from the activated side chain and B-lactam nucleus. Hydrolysis product is understood to be the corresponding acid of the activated side chain.

In the context of the invention enzymatic activity is defined as the volumetric productivity per quantity of dissolved or immobilised enzyme at defined conditions during the enzymatic acylation reaction. Preferably enzymes are applied in immobilised form and the enzymatic activity is defined per quantity of immobilised enzyme.

The volumetric productivity in an acylation reaction can be expressed as the molar quantity of β-lactam antibiotic formed in the acylation reaction at defined conditions during the reaction per unit volume and per unit time.

The S/H ratio is a function of, amongst other things, the concentration of the reactants, the temperature,the pH and the enzyme. It is therefore important to indicate in what conditions an S/H ratio is determined.

A process for the enzymatic preparation of a β-lactam antibiotic from a β-lactam nucleus and an activated side chain with the aid of a penicillin acylase mutant is known from the International patent application WO 98/20120. This publication discloses that a β-lactam antibiotic can be prepared by carrying out an enzymatic acylation reaction in which a β-lactam nucleus and an activated side chain are contacted with each other in the presence of a penicillin G acylase. The β-lactam antibiotic is formed by coupling the side chain to the nucleus. Activated side chains usually are amides or esters of the side chain.

International patent application WO 98/20120 discloses that the enzymatic activity and selectivity for the acylabon reaction can be changed by mutating the penicillin G acylase enzyme at one or more amino acid positions. Specifically, good penicillin G acylases result from mutations at the amino acid positions 142 and 146 on the α-subunit and at amino acid positions 24, 56 or 177 on the β-subunit. Particularly, the mutation in position β24, whereby the L-phenylalanine originally present in the 24 position is replaced by L-alanine (F24A), appears to produce a significantly higher yield in the synthesis of penicillins and cephalosporins with the aid of an activated side chain in the form of an ester.

We found that if an amide is used in combination with the F24A mutant the S/H ratio is relatively high, but the enzymatic activity is so low that the use of this mutant is economically less attractive.

We found it is attractive to use amides as activated side chains, because amides are chemically more stable than esters. A greater chemical stability is understood to mean that hydrolysis of the activated side chain to form the corresponding acid and/or the chemical racemisation of the side chain are significantly lower for amides than for esters. Phenylglycine amide, for example, is around one thousand times more stable than phenylglycine methyl ester at equal temperature and pH.

The object of the invention is therefore to provide a mutated penicillin acylase which, when used in a process for the enzymatic preparation of a β-lactam antibiotic from a β-lactam nucleus and an amide as activated side chain, results in a relatively high S/H ratio and relatively high activity.

Surprisingly, it has been found that a higher S/H ratio is obtained in a process for the preparation of a β-lactam antibiotic whereby, in the presence of a penicillin acylase and with the aid of an activated side chain a β-lactam nucleus is acylated as a mutated penicillin acylase, which acylase has at least one amino acid substitution in a position corresponding to position 145 on the α-subunit of a penicillin acylase originating from E. coli. In particular, a higher S/H ratio is obtained in said process in the presence of a mutated penicillin acylase in which L-Arganine has been replaced in said 145 position by L-Leucine (R145L), L-Lysine (R145K) or L-Cysteine (R145C).

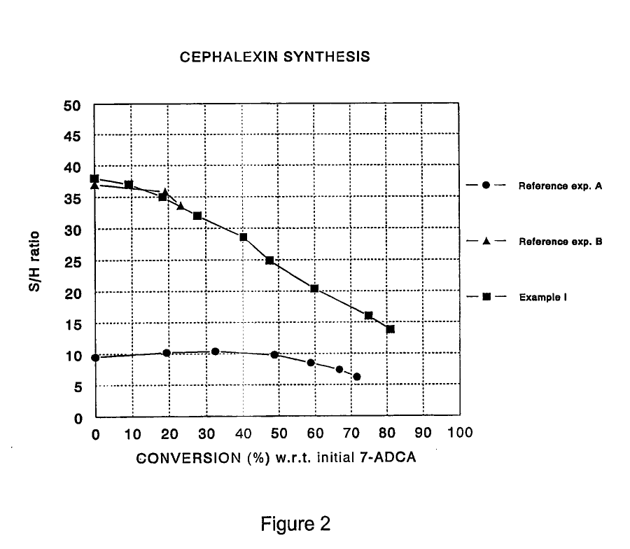

It is known that positions in a penicillin acylase which correspond to a particular position in a penicillin acylase of another wild type can be found by, for example, aligning the amino acid sequences. International patent application WO96/05318, for example, describes how amino acid sequences of penicillin acylases are aligned and shows amino acid sequences for penicillin acylases originating from E. coli, Alcaligenes faecalis, Kluyvera citrophila, Arthrobacter viscosis and P. rettgeri. It is indicated which amino acid positions In for example E. coli correspond with amino acid positions in A. faecalis (see FIG. 2 of WO96/05318).

The R145L, R145K and R145C mutants, when used in a process for the enzymatic preparation of a β-lactam antibiotic from a β-lactam nucleus and an amide as activated side chain, result in an initial S/H ratio at least twice as high as the initial S/H ratio which is found when a wild-type E. coli penicillin G acylase is used, and has an is enzymatic activity amounting to more than 1% of the activity of the wild-type E. coli penicillin G acylase. The invention also relates to a mutated penicillin acylase which, when used in a process for the enzymatic preparation of a β-lactam antibiotic from a β-lactam nucleus and an amide as activated side chain with the aid of the mutated penicillin acylase, results in an initial S/H ratio at least twice as high as the initial S/H ratio which is found when a wild-type E. coli penicillin G acylase is used under the same reaction conditions and which has an enzymatic activity amounting to at least 1% of the activity of the wild-type E. coli penicillin G acylase under the same reaction conditions. Preferably, the mutated penicillin acylase according to the invention is an E. coli penicillin acylase.

The invention in addition relates to a process for the enzymatic preparation of a β-lactam antibiotic from a β-lactam nucleus and an activated side chain with the aid of a mutated penicillin acylase according to the invention.

In a preferred embodiment the invention relates to a process for the preparation of a β-lactam antibiotic whereby, in the presence of a penicillin acylase according to the invention, a β-lactam nucleus is acylated with the aid of an activated side chain In the form of an amide.

In a preferred embodiment the mutated penicillin acylase is an acylase which, when used in a process for the enzymatic preparation of a β-lactam antibiotic from a β-lactam nucleus and an amide as activated side chain with the aid of the mutated acylase, results In an at least 3 and preferably 4 times higher initial S/H ratio than the initial S/H ratio which is found when a wild-type E. coli penicillin G acylase is used.

The enzymatic activity is preferably at least 2%, more preferably at least 5% relative to the activity of the wild-type Ecoli penicillin G acylase.

International patent application WO96/05318 refers to penicillin acylase mutants αM143V, βL56G and βL177S in connection with an improved S/H ratio for the synthesis of Ampidllin from D-phenylglycine amide and 6-aminopenicillanic acid. However, the S/H ratios cited are not a factor of 2 higher than those for a wild-type E. coli penicillin acylase but are significantly lower.

The conversion to be achieved in an acylation reaction can be expressed as the molar quantity of β-lactam antibiotic formed in the acylation reaction at a particular moment during the reaction per molar quantity of reactant used, where the reactant may be either the β-lactam nucleus or the (activated) side chain. In the context of the invention conversion is defined as the quantity of β-lactam antibiotic formed in the acylabon reaction (in moles) per quantity of β-lactam nucleus used (in moles).

During an enzymatic acylation reaction the S/H ratio generally decreases. The conversion generally first increases and later decreases. The S/H ratio is a function of the conversion, amongst other things. The S/H ratios of different penicillin acylases are preferably compared at equal conversion. They are most usually compared at 0% conversion, the so-called initial S/H ratio, which thus is a measure of the S/H ratio. The initial S/H ratio is understood to be the S/H ratio at 0% conversion, hence at time t=0. The initial S/H ratio can be determined with sufficient accuracy by carrying out the acylation reaction until a sufficiently high conversion is reached, at least 30%, preferably at least 50%, and then constructing a graph of the S/H ratio versus conversion and extrapolating it to 0% conversion. It is desirable to determine the initial S/H ratio through extrapolation, since this improves the accuracy of the determination of the initial S/H ratio. For accurate determination it is desirable to have sufficient data points, for instance, at least three data points, which should preferably represent a difference In conversion of at least 5% and wherein preferably none of the measuring points are located where the conversion is less than 10%

During an enzymatic acylation reaction the volumetric productivity and the enzymatic productivity generally decrease over time. The volumetric productivity and the enzymatic activity are a function of the conversion. The enzymatic activities of different penicillin acylases are preferably compared at equal conversion. They are most usually compared at 0% conversion, the so-called initial enzymatic activity, which thus is a measure of enzymatic activity. The initial enzymatic activity is understood to be the enzymatic activity at 0% conversion, hence at time t=0. The initial enzymatic activity can be determined with sufficient accuracy by carrying out the acylation reaction until a certain high conversion is reached, preferably at least 30%, more preferably at least 50%, and then constructing a graph of the enzymatic activity versus the conversion and extrapolating it to 0% conversion. It is desirable to determine the initial enzymatic activity through extrapolation, since this improves the accuracy of the determination of the initial enzymatic activity. For accurate determination it is also desirable to have sufficient data points, preferably, at least three data points, which data points preferably represent a difference in conversion of at least 2% and wherein preferably none of the measuring points should be located where the conversion is less than 1%.

Suitable reaction conditions for the performance of an enzymatic acylation reaction in which a mutated or unmutated penicillin acylase is used are known to one skilled in the art.

The molar ratio of activated side chain to β-lactam nucleus, i.e. the total quantity of activated side chain added divided by the total quantity of β-lactam nucleus added, both expressed in moles, may vary between wide limits. Preferably the molar ratio is between 0.5 and 2.0, in particular between 0.7 and 1.8.

The temperature at which the enzymatic acylation reaction is carried out is generally lower than 40° C., preferably between −5 and 35° C. The pH at which the enzymatic acylation reaction is carried out generally lies between 5.5 and 9.5, preferably between 6.0 and 9.0.

Preferably the reaction is almost completely terminated when the maximum conversion Is all but reached. A suitable embodiment to terminate the reaction is lowering the pH, preferably to a value between 4.0 and 6.3, in particular between 4.5 and 5.7. Another suitable embodiment is lowering the temperature of the reaction mixture as soon as maximum conversion is achieved. A combination of both embodiments is also possible.

In the context of the invention, a decrease in pH can be accomplished for example by the addition of an acid. Suitable acids are for example mineral acids, in particular sulphuric acid, hydrochloric acid solution or nitric acid and carboxylic acids, for example acetic acid, oxalic acid and citric acid. An increase in pH can be accomplished for example by the addition of a base. Suitable bases are for example inorganic bases, in particular ammonia, potassium hydroxide or sodium hydroxide solution and organic bases, for example triethylamine and D-phenylglycine amide. Preferably ammonia is applied.

The enzymatic acylation reaction can be carried out in water. If desired, the reaction mixture may also contain an organic solvent or a mixture of organic solvents, preferably less than 30 vol. %. Examples of organic solvents that may be applied are alcohols with 1-7 C-atoms, for example a monoalcohol, in particular methanol or ethanol; a diol, in particular ethylene glycol or a triol, in particular glycerol.

The enzymatic acylation reaction is preferably carried out as a batch process. If desired, it Is also possible to carry out the reaction continuously. Suitable examples of β-lactam nuclei that can be used in the process according to the invention are penicillin derivatives, for example 6-APA, and cephalosporin derivatives, for example a 7-aminocephalosporanic acid with or without a substituent on the 3-site, for example 7-ACA, 7-ADCA, 7-aminodeacetylcephalosporanic acid (7-ADAC), 7-amino-3-chloro-ceph-3-em-4-carboxylic acid (7-ACCA) and 7-amino-3-chloro-8-oxo-1-azabicyclo[4.2.0]oct-2-ene-2-carboxylic acid.

Side chains in the context of the present invention may include any suitable compounds that can be attached to the 6-penam or 7-cephem position of a β-lactam nucleus, resulting in an antibiotically active compound. Phenylglycine, p-hydroxyphenylglycine and dihydrophenylglycine are examples of suitable side chains. As an activated side chain in the (enzymatic) acylation reaction use may be made of for example an amide (primary, secondary, tertiary) or an ester of said side chains, for instance an amide or ester of phenylglycine, p-hydroxyphenylglycine or dihydrophenylglycine, preferably an amide or a lower alkyl (1-4C) ester, for example a methyl ester. The activated side chain may also include salts of the esters or amides. Preferably, phenylglycine amide is used as an activated side chain.

β-lactam antibiotics that are preferably prepared by the process according to the invention are amoxicillin, ampicillin, cephalexin, cefadroxil, cephradine and cefaclor.

Mutants of penicillin acylases can be made by starting from any known penicillin acylase. Microorganisms from which penicillin acylase enzymes can be obtained are for example Acetobacter, in particular Acetobacter pasteutianum, Aeromonas, Alcaligenes, In particular Alcaligenes faecalis, Aphanocladium, Bacillus sp., in particular Bacillus megaterium, Cephalosporium, Escherchia, in particular Escherichia coli, Flavobactedum, Fusarium, in particular Fusarium oxysporum and Fusadium solani, Kluyvera, Mycoplana, Protaminobacter, Proteus, in particular Proteus rettgari, Pseudomonas and Xanthomonas, in particular Xanthomonas citrii.

Mutated penicillin acylases can be prepared in any known manner. A suitable process is for example a process in which a polymerase chain reaction (PCR) is used to Introduce a mutation in the DNA that codes for a penicillin acylase. In the PCR process a mutation is introduced, with the aid of a synthetic oligonucleotide, in the desired site in a DNA sequence that codes for a penicillin acylase. Use may be made of for example the oligonucleotide

5′-TGCCAGATTATCGATTTCGCTAGT

ACTATCAGAGAACAAGTTTGCCAT-3′

in which the underlined codon codes for L-leucine, for the purpose of introducing a mutation in position 145 of the a-subunit of an E. coli penicillin acylase with the codon for L-Arginine being replaced in that position by a codon for L-Leucine. Oligonucleotides as described above, in which the underlined codon is replaced by ACA or CTT can be used in order to Introduce in the same position a codon for L-Cysteine or L-Lysine, respectively.

After the introduction of the mutation in the DNA sequence, the obtained DNA sequence may be cloned into a vector. The vector may subsequently be used for transforming a host cell. The host cell is then cultivated under conditions suitable for expression of the mutated penicillin acylase.

The invention also relates to a nucleic acid sequence that codes for the mutated penicillin acylase according to the invention and to an expression vector containing the nucleic acid sequence according to the invention with a functionally linked promotor that is suitable for expression of the nucleic acid sequence in a host cell. The invention also relates to a host cell containing the expression vector and to a process for the preparation of a mutated penicillin acylase according to the invention in which the host cell Is cultivated under conditions suitable for production of the mutated penicillin acylase. E. coli is preferably used as the host cell.

Preferably a penicillin acylase is applied in immobilised form, since the immobilised enzyme can then be readily separated and recycled.

The invention will be elucidated on the basis of the examples without being limited thereby.

Nomenclature of amino acids

| Amino acid | 3-letter abbreviation | 1-letter abbreviation |
|---|---|---|
| L-alanine | Ala | A |
| L-cysteine | Cys | C |
| L-asparaginic acid | Asp | D |
| L-glutaminic acid | Glu | E |
| L-phenylalanine | Phe | F |
| L-glycine | Gly | G |
| L-histidine | His | H |
| L-isoleucine | Ile | I |
| L-lysine | Lys | K |
| L-leucine | Leu | L |
| L-methionine | Met | M |
| L-asparagine | Asn | N |
| L-proline | Pro | P |
| L-glutamine | Gl | Q |
| L-arginine | Arg | R |
| L-serine | Ser | S |
| L-threonine | Thr | T |
| L-tyrosine | Tyr | Y |
| L-valine | Val | V |
| L-tryptophan | Trp | W |

List of abbreviations

| | |
|---|---|
| 7-ADCA | 7-aminodesacetoxycephalosporanic acid |
| 6-APA | 6-aminopenicillanic acid |
| AMPI | ampicillin |
| CEX | cephalexin |
| PG | D-phenylglycine |
| PGA | D-phenylglycine amide |
| PGM.HCL | D-phenylglycine methyl ester HCl salt |
| HPGM | D-p-hydroxyphenylglycine methyl ester |

### EXAMPLES AND COMPARATIVE EXPERIMENTS

Penicillin Acylase (Wild Type)

Assemblase™ is an immobilised E. coli penicillin acylase of E. coli ATCC 1105 as described in WO-A-99/20786 and in WO-A-97/04086. The acylase was immobilised on spherules as described in EP-A-222462, utilising gelatin and chitosan as gelating compounds and glutaraldehyde as a crosslinker.

The activity of the E. coli penicillin acylase produced is determined by the quantity of enzyme that is added to the activated spherules and amounted to 3 ASU/g dry weight. 1 ASU (Amoxicillin Synthesis Unit) is defined as the quantity of enzyme needed to produce 1 g of Amoxicillin.3H₂O per hour from 6-APA and HPGM (at 20° C.; 6.5 mass % 6-APA and 6.5 mass % HPGM).

### Example 1

Mutated Penicillin Acylase (R145L)

Preparation of Mutants

In a polymerase chain reaction (PCR) a DNA fragment was amplified by thermocycling. For this purpose use was made of 2 primers each of which was complementary to the DNA (E. coli) of one of the two ends of the fragment (template) which was amplified. After the primers had been bound to the template the DNA polymerase was able to bind to the primer template complex and the DNA was amplified. During thermocycling the following processes took place:

Step 1: Incubation at 94° C.: the double strand template melts and single strand template DNA is formed.

Step 2: Incubation at 55° C.: The primers adhere to the complementary DNA of the template.

Step 3: Incubation at 72+ C.: The polymerase binds to the primer template complex and amplifies the DNA.

By repeating these 3 steps a number of times the fragment was amplified on the template DNA which was flanked by the sequences that are complementary to the primer sequence.

The αR145L mutation was made by the PCR described above. A fragment of 305 base pairs was amplified with the aid of the following reaction mixture:

1 μl primer R145Lfw (100 ng/μl): 5′-GMGTGCTTGGCAAA-3′

1 μl primer R145Lrv (100 ng/μl):

5′-TGCCAGATTATCGATTTCGCTAGT

ACTATCAGAGAACAAGTTTGCCAT-3′

(in which the underlined codon codes for L-leucine)
1 μl plasmid DNA, pEC (˜20 ng/μl stock solution)
2 μl dNTPs (10 mM stock solution)
10 μl 10×Pwo buffer
1 μl Pwo polymerase
84 μl H₂O

The following temperature programme was used to heat the reaction mixture: 1 cycle of 1 min. at 94° C., 25 cycles of: 1 min at 94° C. followed by 1 min. at 55° C. followed by 1 min. at 72° C., 1 cycle of 5 min. at 72° C.

The PCR product and the plasmid pEC were subsequently cut with the restriction enzymes ClaI and EcoRV. Both products were then applied on an agarose gel and purified with the aid of the Quiaex kit from Quiagen. After purification both fragments were ligated. This was done using the following reaction mix.

10 μl PCR product cut with Eco RV and ClaI in H₂O (300 ng DNA in total)

1 μl plasmid DNA cut with EcoRV and ClaI in H₂O (100 ng DNA in total)

2 μl ligation buffer

1 μl ligase

6 μl H₂O

The reaction was carried out at room temperature for 16 hours. Subsequently 10 μl of this reaction mix was used to transform CaCl₂ competent E. coli HB101. Transformation took place through standard methods described in Sambrook et al, ‘Molecular Cloning: a Laboratory Manual’, Cold Spring Harbor Laboratory Press, Cold Spring Harbor, 1989. All enzymes and buffers used were from Boehringer Mannheim.

By sequencing it was verified that the mutant obtained contained the desired nucleotide sequence.

Purification Protocol

The recombinant enzyme was obtained as follows. E. coli with the plasmid pEC containing the gene for penicillin acylase with the relevant mutation was cultivated at 17° C., 150 rpm, in LB medium with 0.1 mM IPTG. The cells were harvested at an optical density at 600 nm (OD600) of 2 by centrifuging at 5000×g for 10 minutes. The pellet was then resuspended in ice-cold buffer A (20% sucrose, 100 mM Tris-HCl, pH 8.0, 10 mm EDTA), in 1/10th of the original volume. Next the cells were centrifuged again for 10 minutes at 5000×g. The pellet was then resuspended in ice-cold buffer B (1 mM EDTA) and centrifuged at 5000×g for 10 minutes. The supernatant is the periplasmatic extract, to which phosphate buffer pH 7.0 was added to a final concentration of 50 mM.

(NH₄)₂SO₄ was added stepwise to the supernatant to a final concentration of 1.5 M after which the extract was centrifuged at 10,000×g for 20 minutes. The supernatant was then placed on a Resource Phe (Amersham-Pharmacia Biotech) and eluted with a linear gradient of 1.5 M-0 M (NH₄)₂SO₄. The final concentration of the purified enzyme was determined by measuring the absorption at 280 nm (A280) (cell length 1 cm) and by using an extinction coefficient of 210.000 M⁻¹ cm⁻¹.

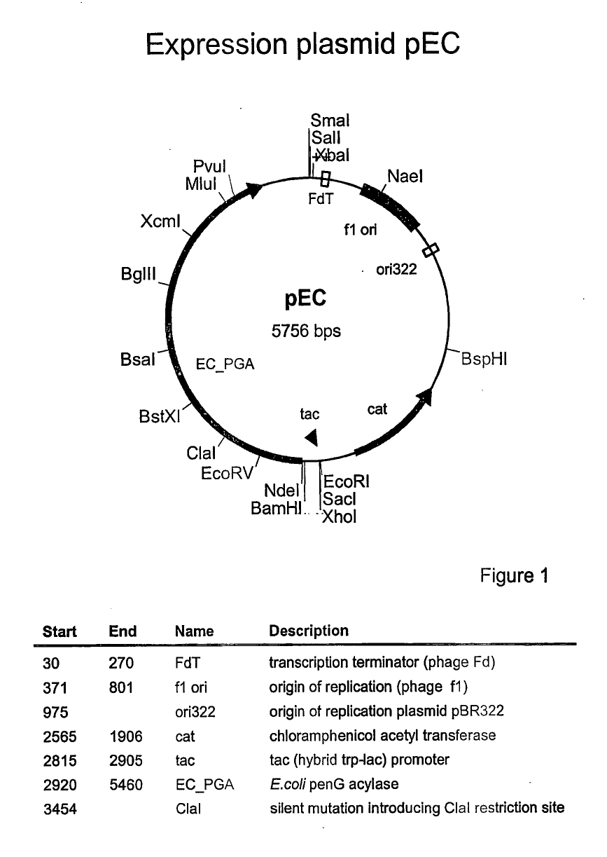

The plasmid pEC is shown in FIG. 1.

Immobilised Pen-G Acylase Mutant R145L

The mutated penicillin acylase (α145L) was immobilised in the same way as described for Assemblase™, except that the mutant R145L was used instead of the wild-type E. coli Pen-G acylase and the enzyme loading was chosen to be a factor of 0.87 (normal loading) and 1.35 (higher loading) higher, respectively, based on immobilised protein.

Reference Experiment A

Immobilised Pen-G Acylase Mutant βF24A

The mutated penicillin acylase (βF24A) was immobilised in the same way as described for Assemblase™, except that the mutant βF24A was used instead of the wild-type E. coli Pen-G acylase. The enzyme loading was chosen to be same, based on immobilised protein.

Synthesis of Cephalexin with Assemblase™ (Immobilised Wild-Type Pen-G Acylase) and PGA

An enzyme reactor (100 ml), with a sieve bottom with 175 μm gauze, was filled with 20 g nett-wet Assemblase™. A preparation reactor (100 ml) was filled with 40.0 ml water (T=2° C.), 0.10 g sodium bisulphite, 10.8 g 7-ADCA (49.6 mmol) and 7.1 g PGA (46.8 mmol). 0.45 g concentrated ammonia was added, after which the suspension was stirred for 15 minutes at T=2° C. The pH was 7.8. Subsequently the suspension was transferred into the enzyme reactor at t=0 with the aid of 10.0 ml water (T=2° C.). At t=0 the stirrer in the enzyme reactor was started. The temperature was kept at T=2° C. After about 90 minutes all 7-ADCA had dissolved; next, a clear solution, apart from solid Assemblase™, was present.

After 166 minutes the pH had risen to 8.80, [PG]=1.10 mass %, the conversion=70 % w.r.t. the initial 7-ADCA amount and the S/H=6.6. The initial enzymatic activity is calculated to be ˜8.2 μmol CEX/(min mg enzyme). The S/H as a function of conversion is shown in FIG. 2.

Reference Experiment B

Synthesis of Cephalexin with Immobilised Pen-G Acylase Mutant βF24A and PGA

A similar enzyme reactor as in reference experiment A was filled with 25.0 g nett-wet of immobilised Pen-G acylase mutant βF24A. A preparation reactor was filled with 40 ml water (2° C.), 0.10 sodium bisulphite, 10.0 g 7-ADCA (45.9 mmol) and 7.2 g PGA (47.5 mmol). 0.45 g ammonia was added. Conditions were further as described in reference experiment A.

After 280 minutes the pH had risen to 7.9, [PG]=0.05 mass %, the conversion=23% w.r.t. the initial 7-ADCA amount and the S/H=33. The Initial enzymatic activity Is calculated to be ˜0.13 μmol CEX/(min mg enzyme). The S/H as a function of conversion is shown in FIG. 2.

### Example 2

Synthesis of Cephalexin with Immobilised Pen-G Acylase Mutant R145L (Normal Loading) and PGA

A similar enzyme reactor as in reference experiment A was filled with 20.0 g nett-wet immobilised Pen-G acylase mutant (R145L, normal loading). A preparation reactor was filled with 47.0 ml water (2° C.), 0.16 g sodium bisulphite, 15.5 g 7-ADCA (70.9 mmol) and 11.5 g PGA (76.0 mmol). Conditions were further as described in reference experiment A.

After 315 minutes the pH had risen to 8.58, [PG]=0.66 mass %, conversion=81% w.r.t. the initial 7-ADCA amount and the S/H =13.8. The initial enzymatic activity is calculated to be ˜0.71 μmol CEX/(min mg enzyme). The S/H as a function of conversion is shown in FIG. 2.

The results (FIG. 2) show that a higher S/H ratio (13.8) in combination with a high conversion (83%) in Cephalexin synthesis is obtained with Pen-G acylase mutant R145L (Example I), compared to the wild type Pen G acylase (Reference experiment A). With Pen-G acylase mutant βF24A (Reference experiment B) only a low conversion (23%) Is obtained. In addition, the initial enzymatic activity of Pen-G acylase mutant R145L Is 8.8% of wild type Pen G acylase, whereas the initial enzymatic activity of Pen-G acylase mutant βF24A is only 1.6% of wild type Pen G acylase.

Reference Experiment C

Synthesis of Ampicillin with Assemblase™ (Immobilised Wild-Type Pen-G Acylase) and PGA

An enzyme reactor (750 ml), with a sieve bottom with 175 μm gauze was filled with 150 g nett-wet Assemblase™.

A preparation reactor (600 ml) was filled with 250 ml water (T=10° C.) and 71.6 g PGA (475 mmol). At T=10° C., 65.8 g 6-APA (300 mmol) was added in small portions in 15 minutes with cooling and with the pH being kept at 7.0 by titration with 6N H₂SO₄. The mixture was stirred for 15 minutes at T=10° C. and subsequently transferred Into the enzyme reactor at t=0 with the aid of 50.0 ml water (T=10° C.). At t=0 the stirrer in the enzyme reactor was started. The pH was kept at 7.0 by titration with 6N H₂SO₄. The temperature was kept at 10° C.

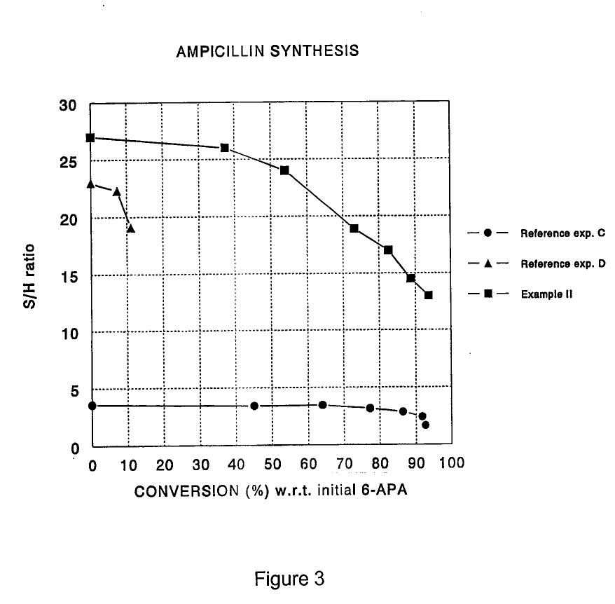

At t=160 minutes the quantity of ampicillin was maximum and the pH was lowered to 5.6 by addition of 6N H₂SO₄. At t=160 minutes the conversion=91-92% w.r.t. the initial 6-APA amount and the S/H=1.67. The initial enzymatic activity is calculated to be ˜1.4 μmol AMPI/(min.mg enzyme). The S/H as a function of conversion is shown in FIG. 3.

Reference Experiment D

Synthesis of Ampicillin with Immobilised Pen-G Acylase Mutant βF24A and PGA

A similar enzyme reactor as in reference experiment C was filled with 95.5 g nett-wet immobilised Pen-G acylase mutant βF24A. A preparation reactor (600 ml) was filled with 200 ml water (T=10° C.), 30.0 g 6-APA (139.0 mmol) and 22.8 g PGA (140 mmol). The pH was 6.7. The mixture was stirred for 15 minutes at T=10° C. and subsequently transferred into the enzyme reactor at t=0 with the aid of 50.0 ml water (T=10° C.). At t=0 the stirrer in the enzyme reactor was started. The pH was kept at 6.7 by titration with 6N H₂SO₄. The temperature was kept at 10° C.

At t=300 minutes the conversion was only 11.1% and the S/H ratio was 19.1. The initial enzymatic activity is calculated to be ˜0.057 μmol AMPI/(min.mg enzyme). The S/H as a function of conversion is shown in FIG. 3.

### Example 3

Synthesis of Ampicillin with Immobilised Pen-G Acylase Mutant R145L and PGA

A similar enzyme reactor as in reference experiment C was filled with 90 g nett-wet immobilised Pen-G acylase mutant R145L (normal loading. Then 59.8 g PGA, 65.8 g 6-APA (300 mmol) and 225 ml water (T=10° C.) were added and stirred for 15 minutes at T=10° C. Then 59.8 g PGA (397 mmol) was added (t=0). After 10 minutes 1.0 g ampicillin trihydrate was added. The temperature being kept at T=10° C. and the pH being kept at 6.9 by titration with 50% acetic acid. A total of 43 ml 50% acetic acid was needed. At t=390 minutes the quantity of ampicillin was maximum and the pH was lowered to 5.6 by addition of 50% acetic acid.

At t=390 minutes the quantity of ampicillin was maximum and the pH was lowered to 5.6 by addition of 50% acetic acid (another 10 ml). The conversion of was 87-88% relative to the quantity of 6-APA used and the S/H ratio was 14.5. The initial enzymatic activity is calculated to be ˜0.94 μmol AMPI/(min.mg enzyme). The S/H as a function of conversion is shown in FIG. 3.

The results show (FIG. 3) that when Pen-G acylase mutant R145L is used in Ampicillin synthesis a higher S/H ratio (14.5) is obtained at high conversion (Example II) compared to wild type Pen G acylase (Reference experiment C): With Pen-G acylase mutant βF24A (Reference experiment D) only a low conversion is obtained. In addition, the initial enzymatic activity of Pen-G acylase mutant R145L is 67% of the wild-type PenG acylase, whereas the initial enzymatic activity of Pen-G acylase mutant βF24A is only 4% of the wild-type PenG acylase.

1. A mutated penicillin acylase which mutated acylase is modified from an unmutated penicillin acylase by an amino acid substitution in a position corresponding to position 145 on the α-subunit of the penicillin G acylase produced by E. coli.

2. The mutated penicillin acylase of claim 1, wherein an L-Arginine at position 145 by L-Leucine, L-Lysine or L-Cysteine.

3. A mutated penicillin G acylase, which catalyzes the preparation of a β-lactam antibiotic from a β-lactam nucleus and an amide as an activated side chain to result in an initial S/H ratio at least twice as high as the initial S/H ratio obtained when said preparation is catalyzed by an unmutated E. coli penicillin G acylase under the same reaction conditions and which has an enzymatic activity at least 1% of the activity of said unmutated E. coli penicillin G acylase under the same reaction conditions.

4. A mutated penicillin acylase of claim 1, which is mutated from an E. coli penicillin acylase.

5. A process to prepare a β-lactam antibiotic which comprises treating a β-lactam nucleus with an activated side chain in the presence of the mutated penicillin acylase, of claim 1.

6. The process of claim 5, the activated side chain is an amide.

7. The process of claim 5, wherein the β-lactam antibiotic is a cephalosporin.

8. The process of claim 5, wherein the β-lactam antibiotic is a penicillin.

9. A nucleic acid molecule comprising a nucleotide sequence that encodes the mutated penicillin acylase of claim 1.

10. An expression vector comprising the nucleotide sequence of claim 9 with a functionally linked promoter for expression of the nucleotide sequence in a host cell.

11. Host cells containing the expression vector of claim 10.

12. A process for preparing a mutated penicillin acylase which is modified from an unmutated acylase by an amino acid substitution in a position corresponding to position 145 on the α-subunit of the penicillin G acylase produced by E. coli which method comprises culturing the host cells of claim 11 under conditions suitable for the production of the mutated acylase.

13. A mutated penicillin acylase of claim 3, which is mutated from an E. coli penicillin acylase.

14. A process to prepare a β-lactam antibiotic which comprises treating a β-lactam nucleus with an activated side chain in the presence of the mutated penicillin acylase of claim 3.

15. The process of claim 14, the activated side chain is an amide.

16. The process of claim 14, wherein the β-lactam antibiotic is a cephalosporin.

17. The process of claim 14, wherein the β-lactam antibiotic is a penicillin.

18. A nucleic acid molecule comprising a nucleotide sequence that encodes the mutated penicillin acylase of claim 3.

19. An expression vector comprising the nucleotide sequence of claim 18 with a functionally linked promoter for expression of the nucleotide sequence in a host cell.

20. Host cells containing the expression vector of claim 19.

21. A process for preparing a mutated penicillin acylase which catalyzes the enzymatic preparation of a β-lactam antibiotic from a β-lactam nucleus and an amide as an activated side chain to result in an initial S/H ratio at least twice as high as the initial S/H ratio obtained when said preparation in catalyzed by an unmutated E. coli penicillin G acylase under the same reaction conditions and has an enzymatic activity of at least 1% of the activity of said unmutated E. coli penicillin G acylase under the same reaction conditions,
which method comprises culturing the host cells of claim 28 under conditions suitable for the production of the mutated acylase.


SEQUENCE LISTING (4 sequences)

SEQ ID NO: 1 - protein, 846 aa, Escherichia coli
MKNRNRMIVNCVTASLMYYWSLPALAEQSSSEIKIVRDEYGMPHIYANDTWHLFYGYGYVVAQDRLFQMEMARRSTQGTVAEVLGKDFVKFDKDIRRNYWPDAIRAQIAALSPEDMSILQGYADGMNAWIDKVNTNPETLLPKQFNTFGFTPKRWEPFDVAMIFVGTMANRFSDSTSEIDNLALLTALKDKYGVSQGMAVFNQLKWLVNPSAPTTIAVQESNYPLKFNQQNSQTAALLPRYDLPAPMLDRPAKGADGALLALTAGKNRETIAAQFAQGGANGLAGYPTTSNMWVIGKSKAQDAKAIMVNGPQFGWYAPAYTYGIGLHGAGYDVTGNTPFAYPGLVFGHNGVISWGSTAGFGDDVDIFAERLSAEKPGYYLHNGKWVKMLSREETITVKNGQAETFTVWRTVHGNILQTDQTTQTAYAKSRAWDGKEVASLLAWTHQMKAKNWQEWTQQAAKQALTINWYYADVNGNIGYVHTGAYPDRQSGHDPRLPVPGTGKWDWKGLLPFEMNPKVYNPQSGYIANWNNSPQKDYPASDLFAFLWGGADRVTEIDRLLEQKPRLTADQAWDVIRQTSRQDLNLRLFLPTLQAATSGLTQSDPRRQLVETLTRWDGINLLNDDGKTWQQPGSAILNVWLTSMLKRTVVAAVPMPFDKWYSASGYETTQDGPTGSLNISVGAKILYEAVQGDKSPIPQAVDLFAGKPQQEVVLAALEDTWETLSKRYGNNVSNWKTPAMALTFRANNFFGVPQAAAEETRHQAEYQNRGTENDMIVFSPTTSDRPVLAWDVVAPGQSGFIAPDGTVDKHYEDQLKMYENFGRKSLWLTKQDVEAHKESQEVLHVQR

SEQ ID NO: 2 - protein, 209 aa, Escherichia coli
EQSSSEIKIVRDEYGMPHIYANDTWHLFYGYGYVVAQDRLFQMEMARRSTQGTVAEVLGKDFVKFDKDIRRNYWPDAIRAQIAALSPEDMSILQGYADGMNAWIDKVNTNPETLLPKQFNTFGFTPKRWEPFDVAMIFVGTMANRFSDSTSEIDNLALLTALKDKYGVSQGMAVFNQLKWLVNPSAPTTIAVQESNYPLKFNQQNSQTA

SEQ ID NO: 3 - protein, 557 aa, Escherichia coli
SNMWVIGKSKAQDAKAIMVNGPQFGWYAPAYTYGIGLHGAGYDVTGNTPFAYPGLVFGHNGVISWGSTAGFGDDVDIFAERLSAEKPGYYLHNGKWVKMLSREETITVKNGQAETFTVWRTVHGNILQTDQTTQTAYAKSRAWDGKEVASLLAWTHQMKAKNWQEWTQQAAKQALTINWYYADVNGNIGYVHTGAYPDRQSGHDPRLPVPGTGKWDWKGLLPFEMNPKVYNPQSGYIANWNNSPQKDYPASDLFAFLWGGADRVTEIDRLLEQKPRLTADQAWDVIRQTSRQDLNLRLFLPTLQAATSGLTQSDPRRQLVETLTRWDGINLLNDDGKTWQQPGSAILNVWLTSMLKRTVVAAVPMPFDKWYSASGYETTQDGPTGSLNISVGAKILYEAVQGDKSPIPQAVDLFAGKPQQEVVLAALEDTWETLSKRYGNNVSNWKTPAMALTFRANNFFGVPQAAAEETRHQAEYQNRGTENDMIVFSPTTSDRPVLAWDVVAPGQSGFIAPDGTVDKHYEDQLKMYENFGRKSLWLTKQDVEAHKESQEVLHVQR

SEQ ID NO: 4 - protein, 209 aa, Escherichia coli
EQSSSEIKIVRDEYGMPHIYANDTWHLFYGYGYVVAQDRLFQMEMARRSTQGTVAEVLGKDFVKFDKDIRRNYWPDAIRAQIAALSPEDMSILQGYADGMNAWIDKVNTNPETLLPKQFNTFGFTPKRWEPFDVAMIFVGTMANLFSDSTSEIDNLALLTALKDKYGVSQGMAVFNQLKWLVNPSAPTTIAVQESNYPLKFNQQNSQTA